(19) United States
(12) Patent Application Publication
(10) Pub. No.: US 2011/0282139 A1
(43) Pub. Date: Nov. 17, 2011

(54) Laser Video Endoscope

(75) Inventor: Martin Uram, Little Silver, NJ (US)

(73) Assignee: Endo Optiks, Inc., Little Silver, NJ (US)

(21) Appl. No.: 12/779,214

(22) Filed: May 13, 2010

(51) Int. Cl. A61B 1/04

(52) U.S. Cl. 600/109

(57) ABSTRACT

A laser video endoscope has a laser guide, an illumination guide and an image guide. These are fiber optical guides which extend through the optical probe and through a hand piece that supports the probe. The hand piece is connected by a first relatively long flexible optical fiber cable to a laser energy source and a source of illumination. By contrast, the image is transmitted from the hand piece to an image presentation site by a camera assembly that is mounted to a hand piece and a relatively long electrical cable. The camera and its electrical cable can be uncoupled from the hand piece and used in a plurality of endoscopic routines. The rest of the product, including the probe and the hand piece, can be disposed of after each medical routine thereby providing assurance of an antiseptic procedure.

### BACKGROUND OF THE INVENTION

This invention relates in general to a medical laser video endoscope and more particularly to one in which the operating probe may be economically disposed after each use.

Laser video endoscopes are known and in particular are described in Applicant's issued U.S. Pat. No. 5,121,740 issued on Jun. 16, 1992 and U.S. Pat. No. 6,997,868 issued on Feb. 14, 2006. The disclosures of those two patents are incorporated herein by reference.

The endoscopes such as the ones described in those two patents, are reused after autoclaving or other sterilization. Reuse occurs in large part because of the expense of the endoscope. The most significant expense factor is the image guide which has a large number of micron size optical fibers. In one endoscope 17,000 fibers were employed thereby providing a 17,000 pixel image.

The image guide currently used costs about $200.00. This is a major incentive for the use of the endoscope after sterilization rather than disposing of the endoscope after each procedure.

This expense factor means that as a practical matter the endoscope will be reused after sterilization rather than disposed of.

However, there is greater security from infection if the probe of the endoscope can be disposed of after each usage instead of being subject to the possibilities of human error in the sterilization process.

Accordingly, it is a key purpose of this invention to provide an endoscope design for which the cost is reasonable enough to permit and encourage disposal of the probe after each use rather then have recourse to sterilization.

It is a related purpose of this invention to provide this cost improvement in a design that maintains a probe design with which the surgeon is familiar and which also maintains the rest of the operating characteristics of the known laser video endoscopes.

It is a further aspect of this invention to provide a laser video endoscope which is less costly than are the current designs.

### BRIEF DESCRIPTION

A laser video endoscope has a laser guide, an illumination guide and an image guide. These are fiber optical guides which extend through the optical probe and through a hand piece that supports the probe. The hand piece is connected by a first relatively long flexible optical fiber cable to a laser energy source and a source of illumination. A camera assembly is connected to the proximal end of the hand piece and coupled to the optical fiber image guide. A relatively long electrical cable transmits an electrical image signal to a site where an image can be provided for the surgery.

The camera and its electrical cable can be uncoupled from the hand piece and used in a plurality of endoscopic routines.

The rest of the product including the probe and the hand piece can be disposed of after each medical routine thereby providing assurance of an antiseptic procedure.

### BRIEF DESCRIPTION OF THE DRAWINGS

FIG. 1 is a schematic illustration of the prior art system extending from the probe 24 to the terminals 12C, 14C and 16C.

FIG. 2 is a schematic illustration of the embodiment of the invention disclosed herein. FIG. 2, like FIG. 1, shows the system extending from the distal probe 30 to proximal terminals 36C, 40C and 42C.

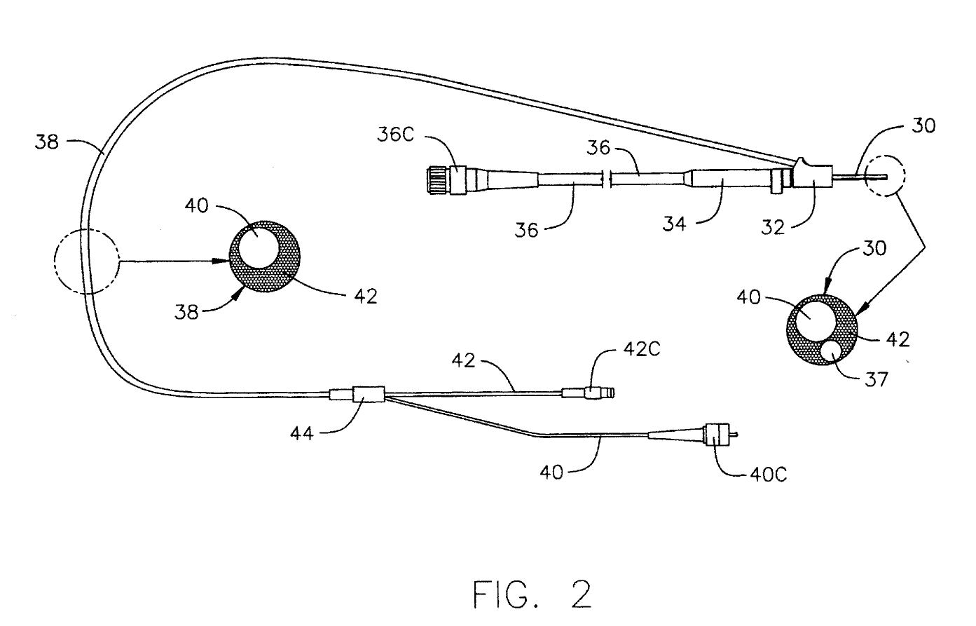

FIG. 3 is a longitudinal view of the camera assembly 34, cable 36 and proximal connector 36C.

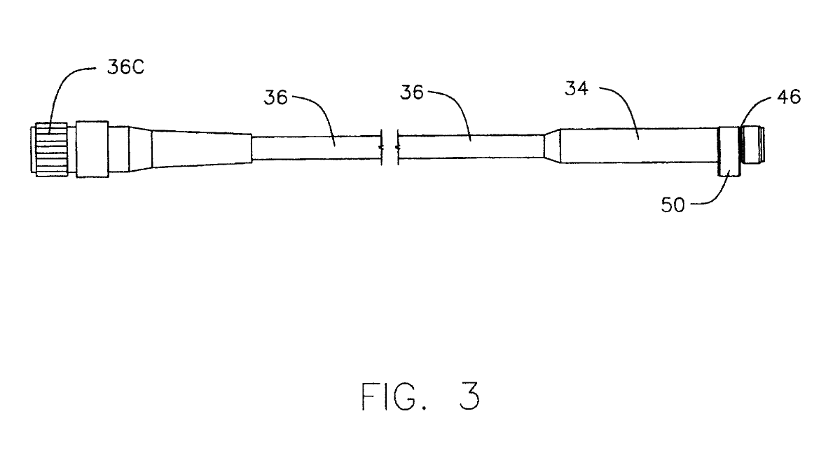

FIG. 4 is a partial longitudinal sectional view of the camera assembly 34 showing camera housing 34, focus ring 50 and laser filter 46. FIG. 4 shows the distal recess 52 for engaging the nose 54 of the hand piece 32.

FIG. 5 is partial sectional view of the hand piece 32.

FIG. 6 is a view similar to that of FIGS. 4 and 5 showing the probe 30 and hand piece 32 assembled and coupled to the camera assembly 34.

### DETAILED DESCRIPTION

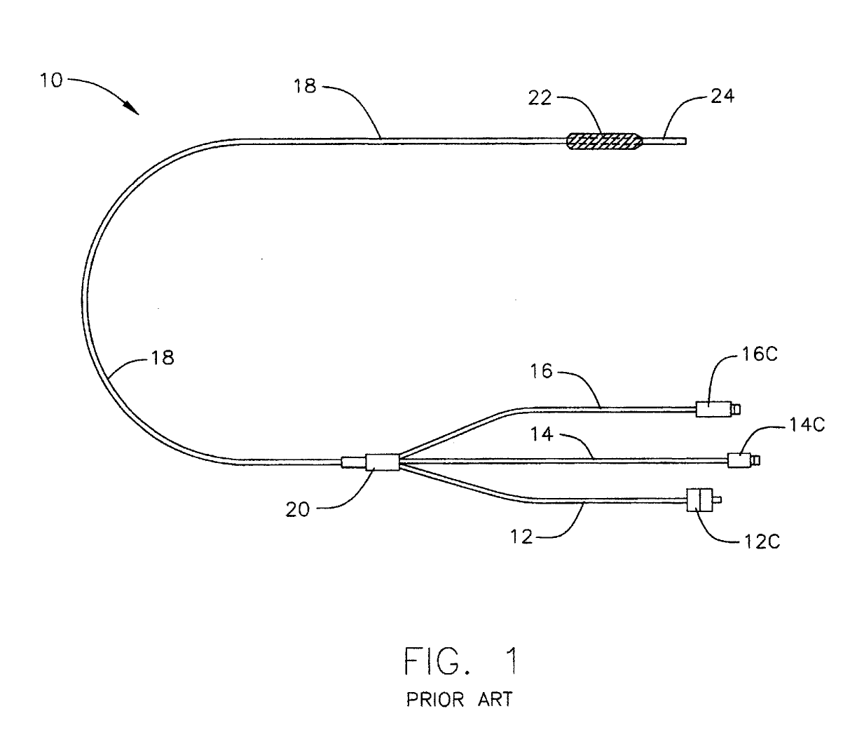

Except for the prior art FIG. 1, the figures are all to a single embodiment.

As shown in FIG. 1, the known laser video endoscopes have an operating probe 24, a hand piece 22, a cable 18 which carries a laser guide 12, an illumination guide 14 and an image guide 16. These are all fiber optic guides which extend from the distal end of the probe 24 to the terminals 12C, 14C and 16C. Distal of the trifurcation zone 20, the fiber optic guides are combined geometrically to provide a minimum diameter cable.

The laser video endoscope of this invention includes the probe 30, a specifically designed hand piece 32 and a camera assembly 34 coupled to the proximal end of the hand piece 36.

The camera assembly 34 is directly connected to the proximal end of the hand piece 32. It has a relatively long electrical cable 36 which extends proximally to a terminal 36C which is coupled to an appropriate display mechanism including a video screen so that the operating surgeon can view the image during the course of manipulating the probe.

A optical guide cable 38 extends in the proximal direction from the hand piece 32 to a bifurcation junction 39. This cable 38 carries the laser and illumination guides 40 and 42 for conveying the laser energy and the illumination energy to the probe 30. At the bifurcation junction 44, the laser guide 40 and illumination guide 42 are separated and terminated at the terminals 40C and 42C for connection to the sources of laser energy and illumination energy. The image carrying electrical cable 36 is about as long as is the optical guide cable 38. Each cable 36, 38 can be as long as required for an installation.

As shown by the coupling mechanism in the camera assembly 34, the optical fibers 40 from the probe 30 and hand piece 32, which carry the image are removably coupled to the camera so that the camera provides an electrical image that is transmitted along the electric cable 36 to the terminal 36C at the base where the video displays are provided. The camera may be any one of a number of known type and may be specially designed to fit the geometry of the camera assembly

Thus by positioning the camera assembly 34 at the hand piece 32, the lengthy and expensive optical image guide is avoided. The camera assembly 34 can be uncoupled from the hand piece 32 so that the relatively expensive camera assembly can be reused. This combination of reuse of the camera assembly 34 and elimination of an extensive length of expensive fiber optic image guide means that disposability of the probe 30 is economically acceptable even though the hand piece 32 and the laser and illumination guides 40, 42 in the cable 38 are also disposed of after each medical routine.

The camera assembly 34 includes a laser filter 46 to protect the camera film from laser energy and to permit the surgeon to observe the operation even when laser pulses are firing. The probe 30 and hand piece 32 are cemented together by a known process.

The camera assembly 34 includes a manually operated spring latch (not shown). The latch is of a known type. It enables readily mounting the camera assembly 34 to the hand piece 32 and, most importantly, removing the camera assembly 34 from the hand piece 32. In addition, the camera assembly 34 includes a focus ring 50 to assure adequate focus of the image provided at the proximal end of the laser fiber image guide 37 in the probe 30 and hand piece 32 onto the image receptors of the camera.

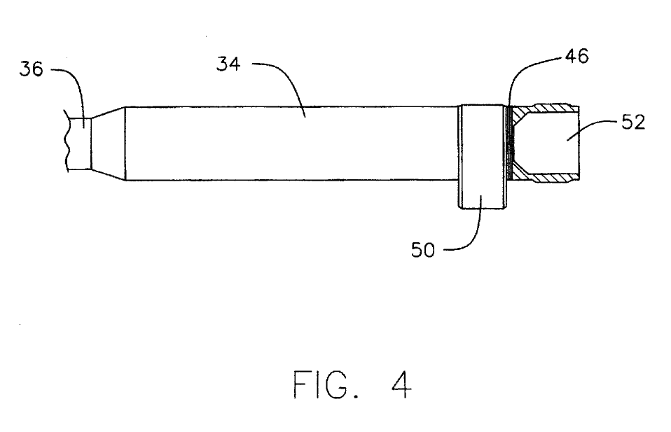

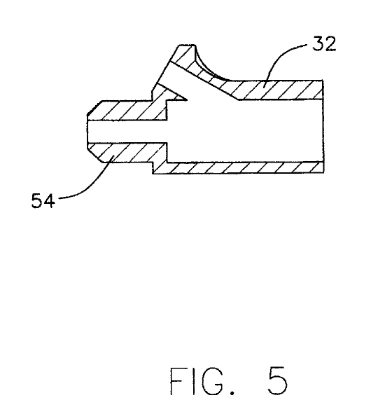

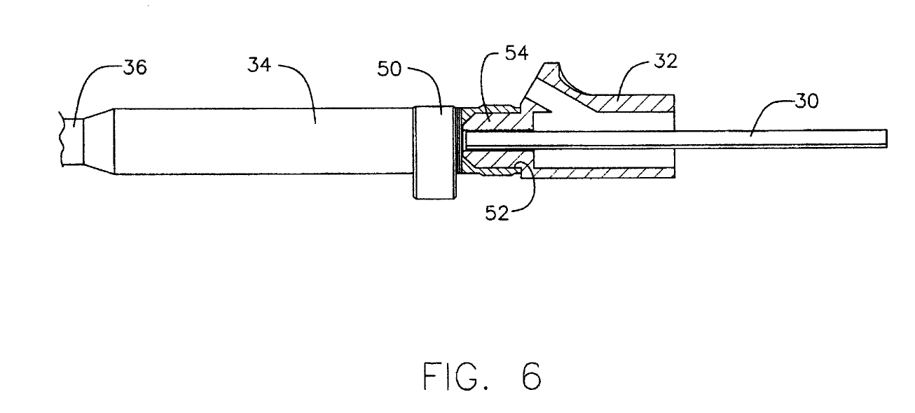

As may be seen from FIGS. 4, 5 and 6, the distal end of the camera assembly has a recess 52 which engages a nose 54 of the hand piece 32. The latch holds the nose 54 in place in the recess 52.

The image guide 37 in the probe 30 and hand piece 32 costs about $8.00. This reduction in cost from about $200.00 to $8.00 is a major factor encouraging disposable use of the endoscope.

A variant on the illustrated embodiment is an arrangement in which the uncoupling at the proximal end of the hand piece 32 will uncouple not only the camera assembly 34 but also the cable 38 so that only the probe 30 and the hand piece 32 would be disposed of between each operation.

It has to be kept in mind that the positioning of the camera assembly 34 at the hand piece 32 permits a standard optical coupling of the image at the proximal end of the optical fiber image guide 37 to the camera assembly 34. It is not feasible to provide a mechanism that will permit coupling and uncoupling the fiber optic image guide 37 at a junction other than the input to the camera. Coupling and uncoupling is otherwise not feasible because of the enormous number of optical fibers which would have to be aligned for such coupling to provide an image that is not degraded or useless.

While the foregoing description and drawings represent the presently preferred embodiments of the invention, it should be understood that those skilled in the art will be able to make changes and modifications to those embodiments without departing from the teachings of the invention and the scope of the claims.

For example, the image guide 37 within the probe 30 and hand piece 32 is a fiber optic bundle of the type normally used. However, there are other means to provide an image guide. One such is the gradient index lens, often referred to as a GRIN lens.

What is claimed is:

1. A surgical endoscope having a hand piece and a probe extending distally from said hand piece, the probe containing an illumination guide, a laser guide and an image guide, the improvement in the combination comprising:
an optical cable containing the illumination guide and the laser guide extending proximally from the hand piece to the laser source and the illumination source,
a camera assembly removably connected to the proximal end of said hand piece and coupled to the image from the image guide in the probe to provide an electrical image at the output of the camera assembly, and
an electrical cable connected to the proximal end of the camera assembly and extending proximally to a terminal adapted to be connected to a mechanism for providing an image,
whereby, after each use of the endoscope, said camera assembly can be detached from said hand piece; and the elements comprising said probe and said hand piece can be disposed of after a single use.

2. The endoscope improvement of claim 1 wherein: said camera assembly incorporates a laser blocking filter and a focusing mechanism,

3. The endoscope improvement of claim 1 wherein: a fiber optic image guide is required only for the distance encompassed by said probe and said hand piece.

4. The endoscope improvement of claim 1 wherein: a fiber optic image guide is required only for the distance encompassed by said probe and said hand piece.

5. The endoscope improvement of claim 1 wherein: said illumination guide, said laser guide and said image cable each terminate proximally in a separate terminal.

6. The endoscope improvement of claim 1 wherein: said illumination guide, said laser guide and said image cable each terminate proximally in a separate terminal.

7. The endoscope improvement of claim 2 wherein: said illumination guide, said laser guide and said image cable each terminate proximally in a separate terminal.

8. The endoscope improvement of claim 4 wherein: said illumination guide, said laser guide and said image cable each terminate proximally in a separate terminal.
